(19) United States
(12) Patent Application Publication
(10) Pub. No.: US 2024/0395412 A1
(43) Pub. Date: Nov. 28, 2024

(54) GENERATING CUMULANT-BASED RISK SCORES FOR DISEASES

(71) Applicant: INTERNATIONAL BUSINESS MACHINES CORPORATION, ARMONK, NY (US)

(72) Inventors: ARITRA BOSE, White Plains, NY (US); Laxmi Parida, Mohegan Lake, NY (US)

(21) Appl. No.: 18/323,640

(22) Filed: May 25, 2023

(51) Int. Cl. G16H 50/30; G16H 50/70

(52) U.S. Cl. CPC G16H 50/30; G16H 50/70

(57) ABSTRACT

An embodiment for generating a cumulant-based continuous variable corresponding to a disease risk. The embodiment may detect multimodal input data associated with an individual. The embodiment may extract interpretable variables from the detected multimodal input data. The embodiment may compute meta-features from the interpretable variables by identifying cumulant-based redescription groups. The embodiment may perform calculating effect sizes for each of the computed meta-features with respect to a target outcome. The embodiment may, based on the calculated effect sizes for each of the computed meta-features, compute a summation of the effect sizes in a hold-out data set with cross-validation to generate a cumulant-based risk score corresponding to the target outcome for the individual.

### BACKGROUND

The present application relates generally to computers, and more particularly, to generating a cumulant-based continuous variable corresponding to a disease risk.

Medical professionals leverage medical data spanning across multiple electronic medical records to treat various types of diseases experienced by their patients. As medical technologies improve, medical professionals continually gain access to an increasing amount of testing and associated medical data. Leveraging this medical data to assess patient risk for various diseases allows medical professionals to better provide most effective options to a given patient that fits their unique circumstances.

### SUMMARY

According to one embodiment, a method, computer system, and computer program product for generating a cumulant-based continuous variable corresponding to a disease risk is provided. The embodiment may include detecting multimodal input data associated with an individual. The embodiment may also include extracting interpretable variables from the detected multimodal input data. The embodiment may further include computing meta-features from the interpretable variables by identifying cumulant-based redescription groups. The embodiment may also include calculating effect sizes for each of the computed meta-features with respect to a target outcome. The embodiment may further include, based on the calculated effect sizes for each of the computed meta-features, computing a summation of the effect sizes in a hold-out data set with cross-validation to generate a cumulant-based risk score corresponding to the target outcome for the individual.

### BRIEF DESCRIPTION OF THE SEVERAL VIEWS OF THE DRAWINGS

These and other objects, features and advantages of the present disclosure will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment; and

FIG. 2 illustrates an operational flowchart for a process of generating a cumulant-based continuous variable corresponding to a disease risk according to at least one embodiment; and

FIG. 3 illustrates an exemplary distribution of cumulant-based risk scores generated according to at least one embodiment.

### DETAILED DESCRIPTION

Detailed embodiments of the claimed structures and methods are disclosed herein; however, it can be understood that the disclosed embodiments are merely illustrative of the claimed structures and methods that may be embodied in various forms. The present disclosure may, however, be embodied in many different forms and should not be construed as limited to the exemplary embodiments set forth herein. In the description, details of well-known features and techniques may be omitted to avoid unnecessarily obscuring the presented embodiments.

It is to be understood that the singular forms “a,” “an,” and “the” include plural referents unless the context clearly dictates otherwise. Thus, for example, reference to “a component surface” includes reference to one or more of such surfaces unless the context clearly dictates otherwise.

Embodiments of the present application relate relates generally to computers, and more particularly, to generating a cumulant-based continuous variable corresponding to a disease risk. The following described exemplary embodiments provide a system, method, and program product to, among other things, detect multimodal input data associated with an individual, extract interpretable variables from the detected multimodal input data, compute meta-features from the interpretable variables by identifying cumulant-based redescription groups, calculate effect sizes for each of the computed meta-features with respect to a target outcome, and based on the calculated effect sizes for each of the computed meta-features, compute a summation of the effect sizes in a hold-out data set with cross-validation to generate a cumulant-based risk score corresponding to the target outcome for the individual. Therefore, the presently described embodiments have the capacity to improve the ability of computers to calculate continuous variables corresponding to a disease risk for an individual, even within environments containing data that is multimodal and involves higher dimension interactions. Presently described embodiments calculate continuous variables corresponding to a disease risk for an individual by generating cumulant-based risk scores using multimodal medical data as an input. Presently described embodiments may embed higher-dimension interactions between multimodal biomarkers into a single quantitative risk score. Presently described embodiments may further develop the generated risk scores based on interpretable and robust multimodal meta-features while preserving interactions between these features. It is envisioned that these generated risk scores may be leveraged in a wide variety of end uses.

As previously described, medical professionals leverage medical data spanning across multiple electronic medical records to treat various types of diseases experienced by their patients. As medical technologies improve, medical professionals continually gain access to an increasing amount of testing and associated medical data. Leveraging this medical data to assess patient risk for various diseases allows medical professionals to better provide most effective options to a given patient that fits their unique circumstances.

However, the treatment of complex diseases requires a comprehensive understanding of a given individual and their medical history from multi-modal data sets spanning across electronic medical records. This may include medical data from various domains, such as, for example, an individual's molecular profiling from whole genomic, transcriptome, proteome sequencing, imaging data, clinical data, and any other useful medical data that may be contained and timestamped within electronic medical records. It thus becomes a significant challenge to parse the many different modalities of medical data to obtain interpretable features and, for case-of-understanding, parse the features data into a single usable risk score that may be used to characterize the patient's likelihood of risk of the disease.

Accordingly, a method, computer system, and computer program product for improved generation of a cumulant-based continuous variable corresponding to a disease risk is provided. The method, system, and computer program product may detect multimodal input data associated with an individual. The method, system, computer program product may extract interpretable variables from the detected multimodal input data. The method, system, computer program product may compute meta-features from the interpretable variables by identifying cumulant-based redescription groups. The method, system, computer program product may then calculate effect sizes for each of the computed meta-features with respect to a target outcome. Thereafter, the method, system, computer program product may, based on the calculated effect sizes for each of the computed meta-features, compute a summation of the effect sizes in a hold-out data set with cross-validation to generate a cumulant-based risk score corresponding to the target outcome for the individual. In turn, the method, system, computer program product has provided for improved methods for using computers to calculate continuous variables corresponding to a disease risk for an individual, even within environments containing data that is multimodal and involves higher dimension interactions. Presently described embodiments calculate continuous variables corresponding to a disease risk for an individual by generating cumulant-based risk scores using multimodal medical data as an input. Presently described embodiments may embed higher-dimension interactions between multimodal biomarkers into a single quantitative risk score. Presently described embodiments may further develop the generated risk scores based on interpretable and robust multimodal meta-features while preserving interactions between these features. It is envisioned that these generated risk scores may be leveraged in a wide variety of end uses.

The present invention may be a system, a method, and/or a computer program product at any possible technical detail level of integration. The computer program product may include a computer readable storage medium (or media) having computer readable program instructions thereon for causing a processor to carry out aspects of the present invention.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

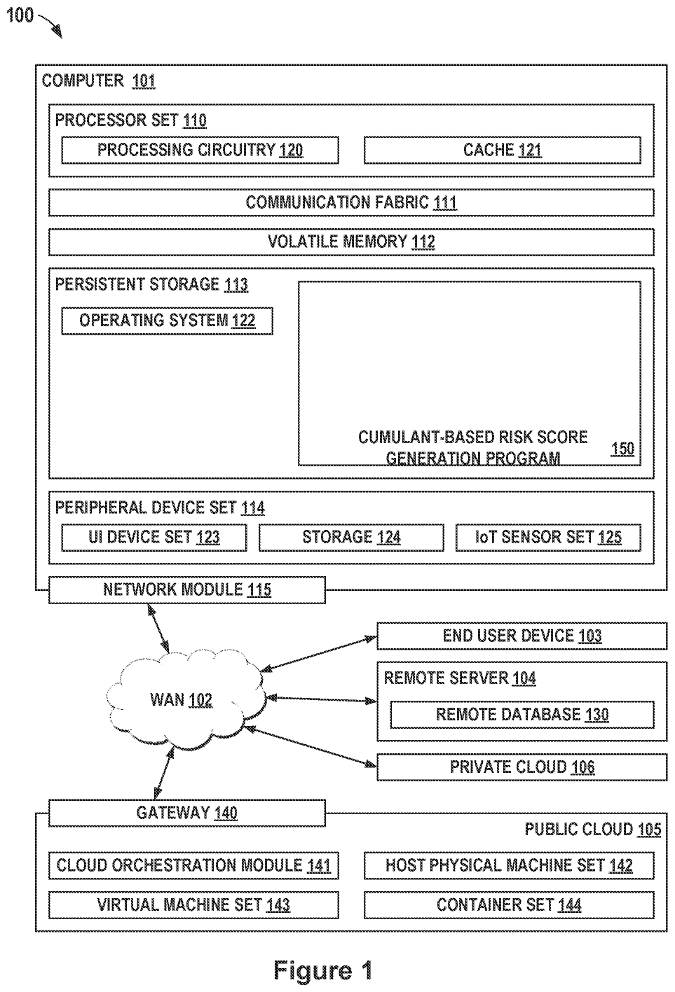

Referring now to FIG. 1, computing environment 100 contains an example of an environment for the execution of at least some of the computer code involved in performing the inventive methods, such as cumulant-based risk score generation program/code 150. In addition to concept cumulant-based risk score generation code 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and cumulant-based risk score generation code 150, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in cumulant-based risk score generation code 150 in persistent storage 113.

COMMUNICATION FABRIC 111 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in cumulant-based risk score generation code 150 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101) and may take any of the forms discussed above in connection with computer 101. EUD 103 typically receives helpful and useful data from the operations of computer 101. For example, in a hypothetical case where computer 101 is designed to provide a recommendation to an end user. this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

According to the present embodiment, the cumulant-based risk score generation program 150 may be a program capable of detecting multimodal input data associated with an individual. Cumulant-based risk score generation program 150 may then extract interpretable variables from the detected multimodal input data. Next, cumulant-based risk score generation program 150 may compute meta-features from the interpretable variables by identifying cumulant-based redescription groups. Cumulant-based risk score generation program 150 may then calculate effect sizes for each of the computed meta-features with respect to a target outcome. Thereafter, cumulant-based risk score generation program 150 may, based on the calculated effect sizes for each of the computed meta-features, compute a summation of the effect sizes in a hold-out data set with cross-validation to generate a cumulant-based risk score corresponding to the target outcome for the individual. Described embodiments thus provide for improved ability of computers to calculate continuous variables corresponding to a disease risk for an individual, even within environments containing data that is multimodal and involves higher dimension interactions. Presently described embodiments calculate continuous variables corresponding to a disease risk for an individual by generating cumulant-based risk scores using multimodal medical data as an input. Presently described embodiments may embed higher-dimension interactions between multimodal biomarkers into a single quantitative risk score. Presently described embodiments may further develop the generated risk scores based on interpretable and robust multimodal meta-features while preserving interactions between these features. It is envisioned that these generated risk scores may be leveraged in a wide variety of end uses.

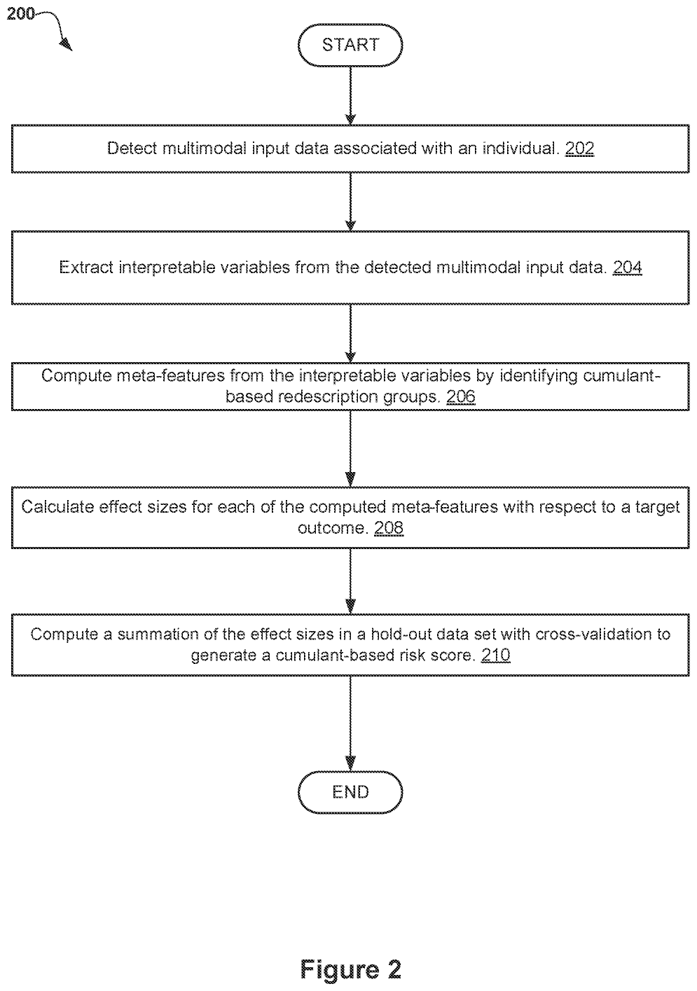

Referring now to FIG. 2, an operational flowchart for a process 200 of process of generating a cumulant-based continuous variable corresponding to a disease risk according to at least one embodiment is provided.

At 202, cumulant-based risk score generation program 150 may detect multimodal input data associated with an individual. In embodiments, the multimodal input data may be in the form of electronic medical records (EMR) including a variety of useful medical data such as, for example, imaging data, clinical data, multi-omics data (genomics, transcriptomics, proteomics, metabolomics, phenomics etc.) or any other useful medical data that may be stored within EMRs. In embodiments, the multimodal input data may include multi-modal binary variables, categorical variables, or continuous variables. In embodiments, the multimodal input data may be stored in any suitable database or repository that may be accessed, and subsequently detected, by the cumulant-based risk score generation program 150. In embodiments, cumulant-based risk score generation program 150 may detect all multimodal input data associated with a target individual. For example, an exemplary cumulant-based risk score generation program 150 may detect multimodal input data for an exemplary individual ‘X’ by accessing an exemplary database ‘D1’ (not shown) which includes EMRs including multimodal medical data such as clinical data for individual ‘X’ including medical history and prescribed medications, imaging data indicating that individual ‘X’ was given an electrocardiogram (ECG), and multi-omics data indicating that individual ‘X’ has a specific genetic variant. This illustrative example involving individual ‘X’ and the associated detected multimodal input data will be discussed in greater detail below throughout the description of process 200.

Next, at 204, cumulant-based risk score generation program 150 may extract interpretable variables from the detected multimodal input data using association studies. Interpretable variables may correspond to any features extracted from multimodal medical data, associated with an individual, which may contribute positively or negatively to the probability of the individual experiencing a specific disease or outcome. In embodiments, cumulant-based risk score generation program 150 may utilize any association study to extract the interpretable features that is suitable or appropriate for the data being processed. For example, cumulant-based risk score generation program 150 may utilize Genome-Wide Association Studies (GWAS) or Phenome-Wide Association Studies (PheWAS) when extracting interpretable variables from genetic data, while it may instead utilize imaging-wide association studies for processing medical imaging data. be Returning to the example used above, at this step, an exemplary cumulant-based risk score generation program 150 may extract, for example, interpretable variables from the detected multimodal input data for the exemplary individual ‘X’. For example, cumulant-based risk score generation program 150 may extract an interpretable variable ‘V1’ from the clinical data for individual ‘X’ which indicates that individual ‘X’ is presently prescribed medication including statins. Cumulant-based risk score generation program 150 may then extract another interpretable variable ‘V2’ from the imaging data (the ECG) performed on individual ‘X’ which corresponds to individual ‘X’ experiencing abnormal T-waves, indicating an abnormal amount of time for the ventricles of the heart to recover from a contraction. Thereafter, cumulant-based risk score generation program 150 may extract yet another interpretable variable ‘V3’ from the multi-omics data indicating that individual ‘X’ has a specific genetic variant in the gene encoding proprotein convertase subtilisin/kexin type 9 (PCSK9) protein.

Next, at 206, cumulant-based risk score generation program 150 may compute meta-features from the interpretable variables by identifying cumulant-based redescription groups. In this step, cumulant-based risk score generation program 150 leverages cumulants to determine higher order relations between the extracted interpretable variables (features) from step 204, as they relate to a target outcome, to generate redescription groups which functionally combine the extracted multiple interpretable variables into a singular meta-feature by using logical regressions. The generated redescription groups help reveal logical relationships among extracted variables. Such relationships may reflect underlying biological pathways implicitly related with connected phenotype patterns. Thus, each of these patterns specify a phenotype, which may be associated with multi-omic data using standard regression methods. Cumulants are statistical measures that may be used to characterize the distribution of random variables. The first few cumulants, such as mean and variance, are commonly used in statistics, however, higher order cumulants can provide additional information about the shape and properties of distributions and can be used to identify groups of observations that share similar distributional properties. Cumulant-based risk score generation program 150 may utilize clustering or other multivariate analysis techniques to group observations that have similar cumulant values. In embodiments, for example, cumulant-based risk score generation program 150 may utilize k-means clustering or hierarchical clustering to identify clusters of observations that have similar mean, variance, skewness, kurtosis, or other higher order cumulants. In embodiments, cumulant-based risk score generation program 150 may use these techniques to identify redescription groups in terms of the extracted interpretable features having similar functions or distribution curves with respect to the probability of being associated with an individual experiencing a target outcome.

In embodiments, each computed meta-feature may correspond to an associated redescription group obtained by cumulant-based risk score generation program 150. At later steps, described in greater detail below, this allows cumulant-based risk score generation program 150 to leverage the redescription groups to ultimately embed higher-dimensional interactions between different multi-modal and multi-omic features. Returning to the example above, at this step, cumulant-based risk score generation program 150 may leverage cumulants to obtain a redescription group that is derived from each of the interpretable variables from step 204 (V1, V2, V3 discussed above) to compute a singular exemplary meta-feature ‘M1’. Computed meta-feature ‘M1’ would thus inherently encompass higher order relations between each of the extracted interpretable variables ‘V1’, ‘V2’, and ‘V3’ combined into a singular new variable in the form of meta-feature ‘M1’ which accordingly includes features derived from the multi-modal data present in the interpretable variables (namely clinical data, imaging data, and omics data). Cumulant-based risk score generation program 150 thus leverages cumulants to allow for consideration higher order relations between any number of features, spanning across multiple modalities, to then compute a singular meta-feature derived from each of the individual features involved. Cumulant-based risk score generation program 150 may then compute a series of meta-features for any of the extracted interpretable variables which are related and may share similar distributions or functions as they relate to a target outcome.

At 208, cumulant-based risk score generation program 150 may perform meta-association studies on the computed meta-features to calculate effect sizes for each of the computed meta-feature with respect to a target outcome. The calculated effect sizes (sometimes referred to as an odds ratio) may be positive, indicating that the meta-feature is associated with an increased probability of an individual experiencing a target outcome, or negative, indicating that the meta-feature is associated with a decreased probability of an individual experiencing a target outcome. In other words, cumulant-based risk score generation program 150 determines how impactful the meta-features are with respect to influencing the probability of the individual experiencing a target outcome. In embodiments, the effect sizes may be represented numerically using odds ratios where any number greater than 1 indicates a positive effect size, and a number less than 1 indicates a negative effect size for a given meta-feature. Because each meta-feature encompasses a singular variable (despite being derived from multiple multi-modal datasets) a singular association study step may be performed to calculate an effect size. In embodiments, for example, the meta-feature association study performed by cumulant-based risk score generation program 150 may involve performing a regression (when one or more variables are continuous) or chi square distribution (when handling categorical variables) to obtain useful curves, p-values, f-statistics, and a basic effect size values. In embodiments, cumulant-based risk score generation program 150 may utilize any suitable regression or chi square distribution based on the variables involved. For example, cumulant-based risk score generation program 150 may utilize a suitable linear logistic regression based on the phenotype or outcome considered. In embodiments, cumulant-based risk score generation program 150 may utilize variable regressions if the outcome variable being considered is continuous, or cumulant-based risk score generation program 150 may utilize binary or multinominal regressions when outcome variables being considered are binary or categorical.

Returning to the example above, at step 208, cumulant-based risk score generation program 150 may perform an association study on the computed meta-feature ‘M1’ to calculate an effect size with respect to an exemplary target outcome ‘TO1’ of experiencing heart disease. Cumulant-based risk score generation program 150 will thus perform a regression to determine the association of meta-feature ‘M1’ with the target outcome ‘TO1’ of experiencing heart disease by utilizing an accessible database ‘D1’ including large amounts of historical datasets related to the features and target outcome. Cumulant-based risk score generation program 150 may calculate, for example, that ‘M1’ has an effect size (odds ratio) of 17.0 with respect to the target outcome ‘TO1’ of experiencing heart disease, indicating that an individual associated with the meta-feature ‘M1’ has an increased probability of experiencing the target outcome ‘TO1’. Because exemplary meta-feature ‘M1’ is derived from interpretable variables involving features related to being prescribed statins, having abnormal T-waves on an ECG, and having an unfavorable specific genetic variant in the gene encoding PCSK9 protein, it is unsurprising that the combination of these features into a singular meta-feature is associated with a positive effect size corresponding to an increased probability in experiencing the outcome of heart disease based on association studies that rely on historical data contained within the accessible database or repository. Cumulant-based risk score generation program 150 may then calculate effect sizes for each additional remaining computed meta-feature associated with the individual associated with the detected multimodal input data.

Thereafter, at 210, cumulant-based risk score generation program 150 may compute a summation of the effect sizes in a hold-out data set with cross-validation to generate a cumulant-based risk score corresponding to the target outcome for the individual. In other words, cumulant-based risk score generation program 150 will aggregate the effect sizes of the meta-features to calculate a risk score for the target outcome and may then plot a density variable. For example, if in addition to ‘M1’ discussed above, cumulant-based risk score generation program 150 had also calculated additional meta-features ‘M2’, ‘M3’, and ‘M4’, then at step 210, cumulant-based risk score generation program 150 may compute a summation of the effect sizes for each of the computed meta-features. With the summed effect sizes considered as a new variable, for example ‘M_{A}’ cumulant-based risk score generation program 150 may utilize a hold-out data set with ‘M_{A}’ as a variable and perform any suitable classification model or techniques to determine the impact of ‘M_{A}’ in predicting the target outcome using the accessible database. In embodiments, to accomplish this, cumulant-based risk score generation program 150 may remove between about 10% to about 20% of the accessible datasets and learn on the remaining datasets. Cumulant-based risk score generation program 150 may then try to predict the risk scores for the target outcome based on the removed percentage of datasets. In embodiments, cumulant-based risk score generation program 150 may then perform cross-validation on the training set to iteratively remove false positives.

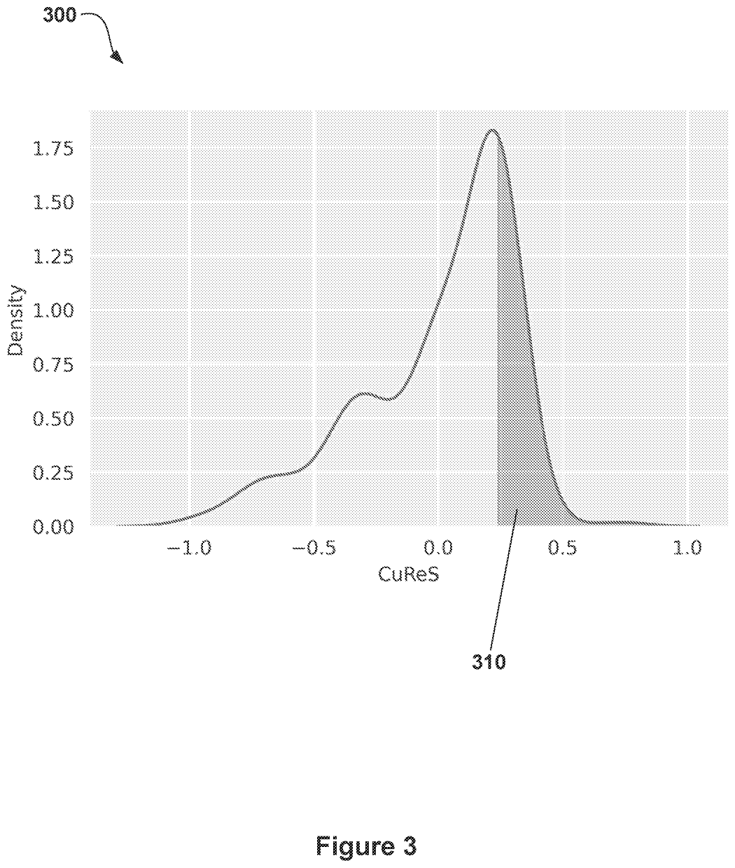

In embodiments, cumulant-based risk score generation program 150 may then plot a distribution using the calculated cumulant-based risk scores with a population density variable, as shown in Plot 300 of FIG. 3. Within plot 300, the calculated cumulant-based risk scores (CuReS) are on the X-axis, and density of population is on the Y-axis. When the risk scores are greater than 0, this indicates a positive risk, while a risk score of less than 0 indicates a negative risk. The density corresponds to a fraction of individuals corresponding to that risk score. Thus, if cumulant-based risk score generation program 150 calculates a cumulant-based risk score for an individual, this risk score may be compared to the corresponding distribution to identify a percentile associated with that individual. For example, using plot 300, cumulant-based risk score generation program 150 may have calculated an individual risk score for individual ‘X’ of 0.45, falling into a shaded region 310 which includes calculated risk scores of around 0.4 to 0.5. Thus, exemplary individual ‘X’ may fall, for example, into the 80^{th} percentile of plot 300, which corresponds to the risk or probability of that individual experiencing the target outcome.

In embodiments cumulant-based risk score generation program 150 may then perform a final step of calculating an area under the curve to calculate phenotypic variance explained by the calculated cumulant-based risk scores with respect to the target outcome. This allows cumulant-based risk score generation program 150 to self-assess the confidence of the model and compare the true positives to false positives by determining how well the calculated cumulant-based risk scores fit the outcomes. In embodiments, the confidence levels obtained by performing this step may be displayed to a user in real-time, or stored in any suitable storage mechanism for future use or displays to a user depending upon the end use of the calculated cumulant-based risk scores.

In embodiments, cumulant-based risk score generation program 150 may further be configured to continuously store calculated cumulant-based risk scores and associated datasets and distributions into a feedback module (not shown) that may be used to employ a feedback loop to allow cumulant-based risk score generation program 150 to continuously expand the amount of available data in order to more effectively calculate future cumulant-based risk scores and improve upon the accuracy of future results.

It will be appreciated that cumulant-based risk score generation program 150 thus provides for improved methods for using computers to calculate continuous variables corresponding to a disease risk for an individual, even within environments containing data that is multimodal and involves higher dimension interactions. Presently described embodiments calculate continuous variables corresponding to a disease risk for an individual by generating cumulant-based risk scores using multimodal medical data as an input. Presently described embodiments may embed higher-dimension interactions between multimodal biomarkers into a single quantitative risk score. Presently described embodiments may further develop the generated risk scores based on interpretable and robust multimodal meta-features while preserving interactions between these features. It is envisioned that these generated risk scores may be leveraged in a wide variety of end uses.

It may be appreciated that FIG. 2 provides only illustrations of an exemplary implementation and does not imply any limitations with regard to how different embodiments may be implemented. Many modifications to the depicted environment may be made based on design and implementation requirements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-based method of generating a cumulant-based continuous variable corresponding to a disease risk, the method comprising:
detecting multimodal input data associated with an individual;
extracting interpretable variables from the detected multimodal input data;
computing meta-features from the interpretable variables by identifying cumulant-based redescription groups;
calculating effect sizes for each of the computed meta-features with respect to a target outcome; and
based on the calculated effect sizes for each of the computed meta-features, computing a summation of the effect sizes in a hold-out data set with cross-validation to generate a cumulant-based risk score corresponding to the target outcome for the individual.

2. The computer-based method of claim 1, further comprising:
calculating an area under the curve to calculate phenotypic variance explained by the cumulant-based risk scores with respect to the target outcome.

3. The computer-based method of claim 1, wherein the input data comprises multi-modal binary, categorical, and continuous variables.

4. The computer-based method of claim 1, further comprising:
generating a distribution which plots the cumulant-based risk scores against a population density variable.

5. The computer-based method of claim 1, wherein extracting the interpretable variables from the detected multimodal input data further comprises:
extracting the interpretable variable using at least one of a regression and a chi square distribution.

6. The computer-based method of claim 1, wherein the generated cumulant-based risk score is obtained by employing a classification model.

7. The computer-based method of claim 6, wherein the employed classification model is a logistic regression.

8. A computer system, the computer system comprising:
one or more processors, one or more computer-readable memories, one or more computer-readable tangible storage medium, and program instructions stored on at least one of the one or more computer-readable tangible storage medium for execution by at least one of the one or more processors via at least one of the one or more computer-readable memories, wherein the computer system is capable of performing a method comprising:
detecting multimodal input data associated with an individual;
extracting interpretable variables from the detected multimodal input data;
computing meta-features from the interpretable variables by identifying cumulant-based redescription groups;
calculating effect sizes for each of the computed meta-features with respect to a target outcome; and
based on the calculated effect sizes for each of the computed meta-features, computing a summation of the effect sizes in a hold-out data set with cross-validation to generate a cumulant-based risk score corresponding to the target outcome for the individual.

9. The computer system of claim 8, further comprising:
calculating an area under the curve to calculate phenotypic variance explained by the cumulant-based risk scores with respect to the target outcome.

10. The computer system of claim 8, wherein the input data comprises multi-modal binary, categorical, and continuous variables.

11. The computer system of claim 8, further comprising:
generating a distribution which plots the cumulant-based risk scores against a population density variable.

12. The computer system of claim 8, wherein extracting the interpretable variables from the detected multimodal input data further comprises:
extracting the interpretable variable using at least one of a regression and a chi square distribution.

13. The computer system of claim 8, wherein the generated cumulant-based risk score is obtained by employing a classification model.

14. The computer system of claim 13, wherein the employed classification model is a logistic regression.

15. A computer program product, the computer program product comprising:
one or more computer-readable tangible storage medium and program instructions stored on at least one of the one or more computer-readable tangible storage medium, the program instructions executable by a processor capable of performing a method, the method comprising:
detecting multimodal input data associated with an individual;
extracting interpretable variables from the detected multimodal input data;
computing meta-features from the interpretable variables by identifying cumulant-based redescription groups;
calculating effect sizes for each of the computed meta-features with respect to a target outcome; and
based on the calculated effect sizes for each of the computed meta-features, computing a summation of the effect sizes in a hold-out data set with cross-validation to generate a cumulant-based risk score corresponding to the target outcome for the individual.

16. The computer program product of claim 15, calculating an area under the curve to calculate phenotypic variance explained by the cumulant-based risk scores with respect to the target outcome.

17. The computer program product of claim 15, wherein the input data comprises multi-modal binary, categorical, and continuous variables.

18. The computer program product of claim 15, further comprising:
generating a distribution which plots the cumulant-based risk scores against a population density variable.

19. The computer program product of claim 15, wherein extracting the interpretable variables from the detected multimodal input data further comprises:
extracting the interpretable variable using at least one of a regression and a chi square distribution.

20. The computer program product of claim 15, wherein the generated cumulant-based risk score is obtained by employing a classification model.
